(19) United States
(12) Patent Application Publication
(10) Pub. No.: US 2019/0046286 A1
(43) Pub. Date: Feb. 14, 2019

(54) Dermatological Laser-Induced Plume Inhibition System and Method

(71) Applicants: Michael P. O'Neil, Dublin, CA (US); Robert E. Grove, Pleasanton, CA (US)

(72) Inventors: Michael P. O'Neil, Dublin, CA (US); Robert E. Grove, Pleasanton, CA (US)

(21) Appl. No.: 16/054,292

(22) Filed: Aug. 3, 2018

Related U.S. Application Data
(60) Provisional application No. 62/542,384, filed on Aug. 8, 2017.

(51) Int. Cl. A61B 90/00; A61B 18/20; A61K 8/81; A61Q 17/04

(52) U.S. Cl. CPC A61B 90/04; A61B 18/203; A61B 2018/00452; A61K 8/817; A61Q 17/04; A61B 90/05

(57) ABSTRACT

A system and method for the reduction or inhibition of laser-induced plumes in dermatological and surgical treatments includes a substantially optically clear physical barrier, that does not degrade due to laser exposure, applied to a treatment area. The dermatological treatment is carried out directly through the barrier. In one embodiment, a barrier may be formed by applying to a treatment area a spray or liquid that polymerizes or cures in situ. In another embodiment, the barrier is pre-formed and placed over the treatment area. The barrier acts to reduce or inhibit the dissipation of the plume that would otherwise result from the laser treatment.

### RELATED APPLICATIONS

This application is related to and claims priority from pending U.S. provisional patent application Ser. No. 62/542,384 filed Aug. 8, 2017, entitled Dermatological Laser-Induced Plume Inhibition System and Method, which is hereby incorporated by reference herein for all purposes.

### FIELD OF THE DISCLOSURE

The invention relates generally to a system and method to reduce or inhibit laser-induced plume in dermatological treatments.

### BACKGROUND

Dermatological laser treatments typically produce plumes of gas, aerosols, smoke and/or ejected particulate matter. This condition also may be known as laser-induced spalling/spallation. Spalling can eject dangerous and/or noxious substances into the immediate environment of the caregiver and patient. The plume may contain infectious aerosols, particles and noxious fumes.

Current practice may use vacuum smoke evacuators to reduce the amount of ejected material. However, the technique is imperfect. Thus, there remains a need for an improved system and method to reduce or inhibit laser-induced plumes.

### SUMMARY

The present disclosure provides a system and method for the reduction or inhibition of laser-induced plumes in dermatological treatments. A substantially optically clear physical barrier that does not degrade due to laser exposure is applied to a treatment area. The dermatological treatment is carried out directly through the barrier. In one embodiment, a barrier may be formed by applying to a treatment area a spray or liquid that polymerizes or cures in situ. In another embodiment, the barrier is pre-formed and placed over the treatment area. The barrier acts to reduce or inhibit the dissipation of the plume that would otherwise result from the laser treatment.

Other benefits and advantages of the present disclosure will be appreciated from the following detailed description.

### DESCRIPTION OF THE DRAWINGS

Exemplary embodiments of an exemplary system and method for the inhibition of laser-induced plumes are shown in the accompanying drawings.

FIG. 1 is a schematic cross-sectional view of a dermatological laser in use with an exemplary embodiment of a plume barrier.

FIG. 2 is a schematic cross-sectional view of laser light applied at a treatment area using an exemplary embodiment of a plume barrier.

### DETAILED DESCRIPTION

Embodiments of the invention and various alternatives are described. Those skilled in the art will recognize, given the teachings herein, that numerous alternatives and equivalents exist which do not depart from the invention. It is therefore intended that the invention not be limited by the description set forth herein or below.

One or more specific embodiments of the system and method will be described below. These described embodiments are only exemplary of the present disclosure. Additionally, in an effort to provide a concise description of these exemplary embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Further, for clarity and convenience only, and without limitation, the disclosure (including the drawings) sets forth exemplary representations of only certain aspects of events and/or circumstances related to this disclosure. Those skilled in the art will recognize, given the teachings herein, additional such aspects, events and/or circumstances related to this disclosure, e.g., additional elements of the devices described; events occurring related to laser-induced plume creation; etc. Such aspects related to this disclosure do not depart from the invention, and it is therefore intended that the invention not be limited by the certain aspects set forth of the events and circumstances related to this disclosure.

The present disclosure provides a system and method for the inhibition of laser-induced plumes in dermatological and surgical treatments. An optically clear physical barrier that does not significantly degrade due to laser exposure is applied directly to a treatment area. The dermatological or surgical treatment is carried out through the barrier. In one embodiment, a barrier may be formed by applying to a treatment area a liquid, gel or wax that polymerizes or cures in situ. The barrier acts to inhibit or prevent the dissipation of the plume into the operating environment that would otherwise result from the laser treatment. The barrier may also provide a means to rapidly convert the smoke, aerosol and airborne ejecta of the plume into a solid form on the inner surface of the barrier thus occupying much less physical space. The “smoke-to-solid” conversion means may be intrinsic to the material properties of the barrier, or may be enhanced by mechanical perforations or slits or similar means to increase the effective surface area of the barrier. A chemical facilitator may also be deployed to absorb gasses or vaporized materials.

In one embodiment a thin, conforming, optically clear barrier may be formed. The liquid may be sprayed, painted, rolled, rubbed, or otherwise applied onto the skin. Once the liquid is polymerized or cured, the resulting barrier may be optically transparent to substantially all commercially available dermatological and surgical treatment lasers. In one embodiment, transparency from about 500 nm to about 1100 nm may be provided. In another embodiment, transparency from 300 nm to at least 10,600 nm may be provided.

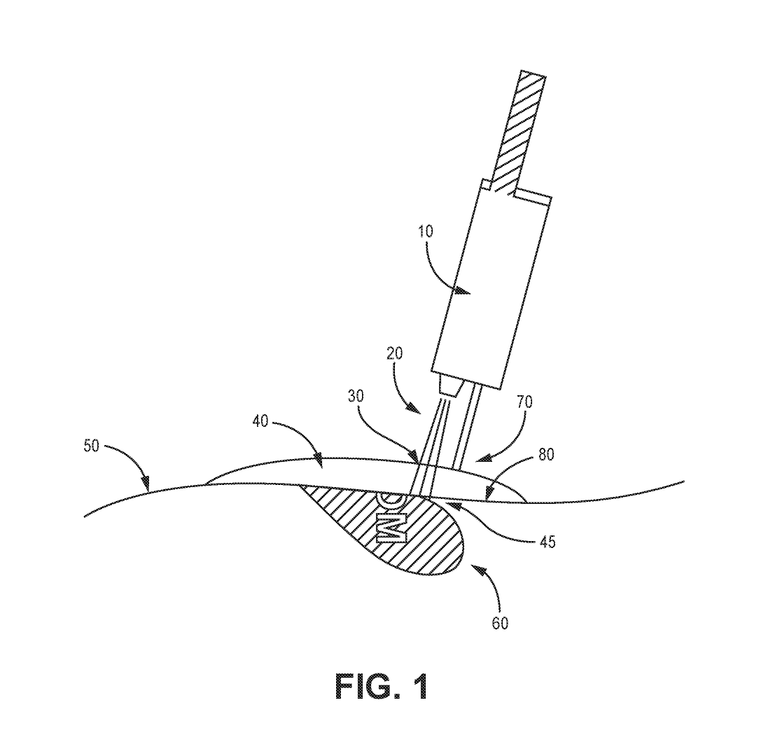

As shown in FIG. 1, a dermatologic or surgical laser 10 fires laser light 20 through optical interface 30 into a transparent plume barrier 40. The laser light 20 passes through a second optical interface 45, through skin 50, and into an area to be treated, such as tattoo 60. A laser hand-piece standoff 70 makes contact with a first, upper surface 30 of the barrier 40. A second surface 80 of barrier 40 makes contact with the upper surface of skin 50. The second surface 80 may be substantially adhesive so as to remain in place during the laser treatment.

In one embodiment, the barrier may be at least in part breathable so as to provide sufficient inhibition of the laser-induced plume while enabling some ambient air to reach the skin. If necessary, an evacuator may be used in combination with a breathable barrier. In another embodiment, the barrier is not breathable.

In performing a dermatological or surgical laser treatment, the laser in one embodiment would be fired through the barrier without significant loss of fluence, energy or power. The barrier would not significantly darken, decompose, or chemically react upon laser exposure.

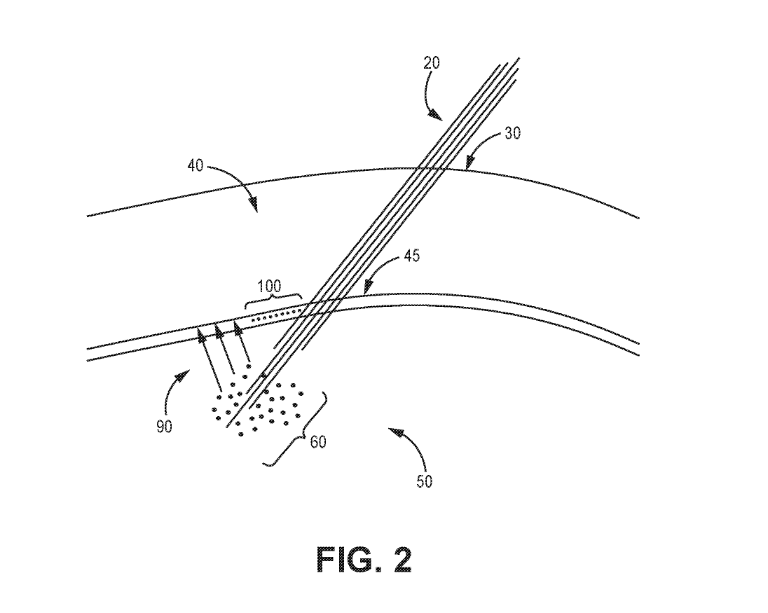

As shown in FIG. 2, in one exemplary embodiment laser light 20 passes through optical interface 30 into a transparent plume barrier 40, through a second optical interface 45, through human skin 50 and into an area to be treated, such as tattoo 60. The plume 90 generated by the laser light 20 may be trapped by the bottom of the transparent barrier 45 and remain contained as particulate or similar matter 100. Alternately, or in addition, particulate matter 100 may be trapped by and within the body of the barrier 40.

After treatment, the barrier in one embodiment may be peeled away in one or more pieces or sheets. Alternately, the barrier may be dissolved in water or another benign solvent (e.g., isopropanol or acetone). Other methods for removing a barrier also may be used. For example, some barriers may “shed” as the underlying skin adds new epithelial cells. The barriers may disappear over time or naturally wear off as the skin heals.

In one embodiment, a removable barrier is temporarily left in place after laser use, e.g., to serve as an aid to healing.

The liquid used to form the barrier may be a silicone-based liquid. In one embodiment, the liquid polymerizes on exposure to water vapor (i.e., similar to a bathtub caulk). In another embodiment, the liquid may be a multi-part system, i.e., similar to NuSil MED-6342 material (a commercially available, two-component system for forming a silicone gel). The system may be pre-mixed and cured before placement over the treatment area. Alternately, the system may be mixed and cured in situ over the treatment area. In yet another embodiment, an evaporating solvent (i.e., vehicle) system may be used.

The barrier may be formed using Scapa Soft-Pro Silicone Gel; NuSil MED2-4420 rapid-cure silicone elastomer; and/or one or more of the two-part polysiloxanes described in U.S. Pat. No. 9,333,223, issued May 10, 2016, entitled “Compositions and Methods for Treating Conditions of Compromised Skin Barrier Function” by Yu, et al., which is hereby incorporated by reference herein for all purposes.

The embodiments described herein provide a durable, convenient, long-lasting coating with skin occlusive benefits. The formulation, composition, film or barrier of the invention provides a means of reducing laser-induced plume.

One embodiment provides a means of reducing laser-induced plume, comprising: applying to a subject's skin a composition comprising a) a reactive reinforcing component; and b) a cross-linking component; in which the cross-linking component facilitates in situ cross-linking of the reactive reinforcing component, such that a film is formed on the skin, thereby reducing, abating or containing said plume.

In another embodiment a barrier is formed using a two-part formulation for application to the skin to reduce laser-induced plume that comprises a) a reactive reinforcing component; and b) a cross-linking component; in which the reactive reinforcing component and the cross-linking component are prevented from coming into contact prior to use; and in which the cross-linking component facilitates in situ cross-linking of the reactive reinforcing component, such that a film is formed on the skin or other tissues.

In one embodiment, formulations are provided for application to the skin to reduce laser-induced plume that comprise a) a reactive reinforcing component; and b) a cross-linking component; in which the reactive reinforcing component has a viscosity of between about 5,000 and about 1,000,000 cSt or cP at 25° C.; and in which the cross-inking component facilitates in situ cross-linking of the reactive reinforcing component, such that a film is formed on the skin.

In one embodiment, formulations are provided for application to the skin to reduce laser-induced plume that comprise a) a reactive reinforcing component; and b) a cross-linking component in which the reactive reinforcing component has a silicone vinyl moiety to functional hydride ratio of between about 1:10 and about 1:100; and in which the cross-linking component facilitates in situ cross-linking of the reactive reinforcing component, such that a film is formed on the skin.

In one embodiment, formulations are provided for application to the skin to reduce laser-induced plume that comprise a) a reactive reinforcing component; and h) a cross-linking component in which the reactive reinforcing component has a silicone vinyl moiety to functional hydride ratio of between about 1.4 and about 1:100; and in which the cross-linking component facilitates in situ cross-linking of the reactive reinforcing component, such that a film is formed on the skin or other areas of the body that are treated by laser surgery.

In one embodiment, films are provided to reduce laser-induced plume prepared by a process comprising the steps of: a) applying a reactive reinforcing component to skin; and b) applying a cross-linking component to the reactive reinforcing component, in which the cross-linking component facilitates in situ cross-linking of the reactive reinforcing component, such that a film is formed on the skin.

In some embodiments, the reactive reinforcing component comprises a reactive constituent and a reinforcing constituent.

In some embodiments, the reactive constituent comprises at least one organopolysiloxane and at least one hydride functionalized polysiloxane. In some embodiments, the reactive constituent comprises at least one high viscosity organopolysiloxane, at least one low viscosity organopolysiloxane and at least one hydride functionalized polysiloxane. In some embodiments, the reactive constituent comprises at least one high viscosity organopolysiloxane or at least one low viscosity organopolysiloxane or a combination thereof.

In some embodiments, the organopolysiloxane is a high viscosity organopolysiloxane or a low viscosity organopolysiloxane or a combination thereof.

In some embodiments, high viscosity organopolysiloxane and the low-viscosity organopolysiloxane are selected from the group consisting of vinyl terminated polydimethylsiloxane; vinyl terminated diphenylsiloxane-dimethylsiloxane copolymers; vinyl terminated poly phenylmethyl siloxane, vinylphenylmethyl terminated vinylphenylsiloxane-phenylmethylsiloxane copolymer; vinyl terminated trifluoropropylmethylsiloxane-dimethylsiloxane copolymer; vinyl terminated diethyl siloxane-dimethyl siloxane copolymer; vinylmethyl siloxane-dimethylsiloxane copolymer, trimethylsiloxy terminated; vinylmethylsiloxane-dimethylsiloxane copolymers, silanol terminated; vinylmethylsiloxane-dimethylsiloxane copolymers, vinyl terminated; vinyl gums; vinylmethylsiloxane homopolymers; vinyl T-structure polymers; monovinyl terminated polydimethylsiloxanes; vinylmethylsiloxane terpolymers; vinylmethoxysilane homopolymers and combinations thereof, including those without vinyl moieties.

In some embodiments, the hydride functionalized polysiloxane is selected from the group consisting of hydride terminated polydimethylsiloxane; polyphenyl-(dimethylhydrosiloxy)siloxane, hydride terminated; methylhydrosiloxane-phenylmethylsiloxane copolymer, hydride terminated; methylhydrosiloxane-dimethylsiloxane copolymers, trimethylsiloxy terminated, polymethylhydrosiloxanes, trimethylsiloxy terminated; polyethylhydrosiloxane, triethylsiloxane, methylhydrosiloxane-phenyloctylmethylsiloxane copolymer; methylhydrosiloxane-phenyloctylmethylsiloxane terpolymer and combinations thereof.

In some embodiments, the reactive reinforcing component further comprises one or more of feel modifiers, spreadability enhancers, adhesion modifiers, diluents, tack modifiers, optics modifiers, particles, and volatile siloxanes.

In some embodiments, the crosslinking component comprises a metal catalyst. In some embodiments, the catalyst is a platinum catalyst. In some embodiments, the catalyst is selected from the group consisting of platinum carbonyl cyclovinylmethylsiloxane complexes, platinum divinyltetramethyldisoloxane complexes, platinum cyclovinylmethylsiloxane complexes, platinum octanaldehyde/octanol complexes and combinations thereof.

The crosslinking component in one embodiment may take the form of a spray-on formulation. As a spray-on formulation, the crosslinking component may have a viscosity between 0.1 to 50 cPs or cSt at 25 C.

The materials and barriers described herein may be used with or without perfluorodecalin (PFD). PFD may help the plume inhibition process by absorbing gas, steam, aerosols and other noxious materials, thereby generally blocking the plume, etc.

In some embodiments, the materials and barriers may be formed using a compound dissolved in a solvent. When the solvent evaporates, a film or barrier is left on the skin that does not require polymerization in situ. Water, acetone, or other volatile solvents may be used. In one embodiment, nitrocellulose is provided in a dissolved form in a pure solvent or a mixture of solvents such as: ethyl acetate, amyl acetate, n-butyl acetate, ethyl alcohol, methyl alcohol, isopropanol or similar. The mixture is applied to the treatment area. When the solvent or solvents are allowed to evaporate, only nitrocellulose remains to coat and cover the treatment area and block or reduce plume formation during laser application.

It should be understood that the foregoing description is only illustrative of the invention. Various alternatives and modifications can be devised by those skilled in the art having the benefit of this disclosure, without departing from the invention. Accordingly, the invention is intended to embrace all such alternatives, modifications and variances.

Certain exemplary embodiments of the disclosure may be described. Of course, the embodiments may be modified in form and content, and are not exhaustive, i.e., additional aspects of the disclosure, as well as additional embodiments, will be understood and may be set forth in view of the description herein. Further, while the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention.

What is claimed is:

1. A method of reducing laser-induced plume, comprising: applying to a subjects skin: (a) a reactive reinforcing component; and (b) a cross-linking component; wherein the cross-linking component facilitates in situ cross-linking of the reactive reinforcing component, such that a film is formed on skin, thereby reducing, abating or containing said plume.

2. The method of claim 1, wherein the reactive reinforcing component has a viscosity of between about 5,000 and about 1,000,000 cSt or cP at 25° C.

3. The method of claim 1, wherein the reactive reinforcing component has a silicone vinyl moiety to functional hydride ratio of between about 1:10 and about 1:100.

4. The method of claim 1, wherein the reactive reinforcing component has a silicone vinyl moiety to functional hydride ratio of between about 1:4 and about 1:100.

5. The method of claim 1, wherein the reactive reinforcing component comprises a reactive constituent and a reinforcing constituent.

6. The method of claim 5, wherein the reactive constituent comprises at least one organopolysiloxane and at least one hydride functionalized polysiloxane.

7. The method of claim 5, wherein the reactive constituent comprises at least one high viscosity organopolysiloxane, at least one low viscosity organopolysiloxane and at least one hydride functionalized polysiloxane.

8. The method of claim 5, wherein the reactive constituent comprises at least one high viscosity organopolysiloxane or at least one low viscosity organopolysiloxane or a combination thereof.

9. The method of claim 6, wherein the organopolysiloxane is a high viscosity organopolysiloxane or a low viscosity organopolysiloxane or a combination thereof.

10. The method of claim 8, wherein the high viscosity organopolysiloxane and the low-viscosity organopolysiloxane are selected from the group consisting of: vinyl terminated polydimethylsiloxane; vinyl terminated diphenylsiloxane-dimethylsiloxane copolymers; vinyl terminated polyphenylmethylsiloxane, vinylphenylmethyl terminated vinylphenylsiloxane-phenylmethylsiloxane copolymer; vinyl terminated trifluoropropylmethylsiloxane-dimethylsiloxane copolymer; vinyl terminated diethylsiloxane-dimethylsiloxane copolymer; vinylmethylsiloxane-dimethylsiloxane copolymer, trimethylsiloxy terminated; vinylmethylsiloxane-dimethylsiloxane copolymers, silanol terminated; vinylmethylsiloxane-dimethylsiloxane copolymers, vinyl terminated; vinyl gums; vinylmethylsiloxane homopolymers; vinyl T-structure polymers; monovinyl terminated polydimethylsiloxanes; vinylmethylsiloxane terpolymers; vinylmethoxysilane homopolymers; and combinations thereof.

11. The method of claim 7, wherein the hydride functionalized polysiloxane is selected from the group consisting of hydride terminated polydimethylsiloxane; polyphenyl-(dimethylhydrosiloxy)siloxane, hydride terminated; methylhydrosiloxane-phenylmethylsiloxane copolymer, hydride terminated; methylhydrosiloxane-dimethylsiloxane copolymers, trimethylsiloxy terminated; polymethylhydrosiloxanes, trimethylsiloxy terminated; polyethylhydrosiloxane, tri ethylsiloxane, methylhydrosiloxane-phenyloctylmethylsiloxane copolymer; methylhydrosiloxane-phenyloctylmethylsiloxane terpolymer; and combinations thereof.

12. The method of claim 1, wherein the reactive reinforcing component includes one or more of feel modifiers, spreadability enhancers, adhesion modifiers, diluents, tack modifiers, optics modifiers, particles, and volatile siloxanes.

13. The method of claim 1, wherein the crosslinking component includes a metal catalyst.

14. The method of claim 13, wherein the metal catalyst is a platinum catalyst.

15. The method of claim 14, wherein the catalyst is selected from the group consisting of platinum carbonyl cyclovinylmethylsiloxane complexes, platinum divinyltetramethyldisoloxane complexes, platinum cyclovinylmethylsiloxane complexes, platinum octanaldehyde/octanol complexes, and combinations thereof.

16. The method of claim 1, wherein the crosslinking component is a spray-on formulation.

17. The method of claim 16, wherein the crosslinking component has a viscosity of between about 0.1 to about 10 cPs or cSt at 25 C.

18. A method of inhibiting laser-induced plume comprising:
(a) forming a barrier by: (i) applying a compound dissolved in a solvent; and (ii) allowing the solvent to evaporate so that the compound remains in situ;
(b) treating with a laser to form said plume, wherein the laser is fired through the barrier and wherein the barrier reduces, abates or contains said plume.

19. The method of claim 18, wherein the compound is substantially transparent.

20. The method of claim 18, wherein the compound is not silicone-based.
